(19) United States
(12) Patent Application Publication
(10) Pub. No.: US 2026/0345784 A1
(43) Pub. Date: Nov. 5, 2026

(54) COMPOUND FOR FORMULATING MOISTURE SENSITIVE ACTIVE INGREDIENTS

(71) Applicant: ROQUETTE FRERES, Lestrem (FR)

(72) Inventors: Baptiste BOIT, LA GORGUE (FR); Philippe LEFEVRE, HAVERSKERQUE (FR); Antoine SALOME, BILLY BERCLAU (FR); Fabrice BUQUET, LESTREM (FR); Carin SIOW, Singapore (SG); Shing Ming OOI, Singapore (SG); Kwan Hang LAM, Singapore (SG)

(21) Appl. No.: 19/123,651

(22) PCT Filed: Oct. 24, 2023
(86) PCT No.: PCT/EP2023/025444

§ 371 Date: Apr. 23, 2025

(30) Foreign Application Priority Data
Oct. 28, 2022 (EP) 22306644.0
Jun. 9, 2023 (EP) 23305925.2

(51) Int. Cl. A61K 47/26; A61K 35/747; A61K 47/36

(52) U.S. Cl. CPC A61K 47/26; A61K 35/747; A61K 47/36

(57) ABSTRACT

Granules of microcrystalline mannitol and of granular starch having a water activity equal to or lower than 0.3. The invention also relates to a process for obtaining thereof. The invention also relates to the use of such granules for formulating or stabilizing moisture-sensitive active ingredients such as probiotics.

### TECHNICAL FIELD

The invention pertains to the field of compounds or excipients for formulating moisture sensitive active ingredients such as probiotics.

### BACKGROUND ART

We have been witnessing for the past few decades a growing trend in probiotic use. Following pharmaceutical forms are used in the production of probiotic food supplements: capsules, powders, drops etc. In the pharmaceutical industry, the most prevalent are capsulated probiotics marketed as dietary supplements. Multidisciplinary teams are tasked with connecting requirements of optimal formulation that will benefit the host's flora with the technological demands in probiotic manufacturing process.

According to the definition from the World Health Organisation (WHO), probiotics represent live organisms that, if applied in optimal amounts, contribute to human health. Probiotics are microorganisms—bacteria and yeast that contribute to the host's health by stimulating the growth of beneficial bacteria, suppressing pathogens via inhibition of mucosal adherence and production of antimicrobials. Microorganisms with probiotic potentials are usually from the genus of Lactobacilli, Bifidobacterium and yeasts.

Water activity, followed closely by storage temperature, is the main factor impacting probiotic stability over the shelf life of the product. Downstream processing and manufacturing of the final product (dietary supplement) needs to happen under strict temperature- and humidity-controlled conditions.

Since the excipients make up a significant proportion of the formulation, it is crucial that these have a low water activity. Establishing a low-water-activity product thus starts with sourcing dry excipients that will be blended with the probiotic. Small amounts of high-water-activity ingredients can be added as long as the total water activity remains low, ideally between 0.2-0.3. The Aw value greater than 0.5 is critical, because that is the point when some probiotic cells e.g., Lactobacillus, degrade more quickly.

The water activity values indicate the free water (water not bound to molecules) which allows biochemical reactions to proceed, and has an impact on maintaining the viability of probiotics. This is different from the moisture content, which is the sum of bound and free water.

Unfortunately, sourcing excipient with low water activity is not sufficient. Indeed, the water activity of the final formula must also remain low upon storage. Otherwise, as soon as the formula will be into contact with moisture, the water activity will increase, and cell viability will be impaired.

It would be thus advantageous to have an excipient which has low water activity without the need for an additional pre-drying step, and which can further maintain the water activity of the probiotic formula into acceptable ranges, in particular of not more than 0.5.

### Technical Problem

It was an object of the present invention to provide an excipient suitable, as is, for formulating moisture-sensitive active ingredients, in particular probiotics.

It was an object of the present invention to provide an excipient that destabilize moisture-sensitive active ingredients to a limited extent, or even stabilize moisture-sensitive active ingredients.

It was an object of the present invention to provide an excipient for formulating moisture-sensitive active ingredients into tablets (in particular chewable tablets), or into powdery compositions (e.g., fillers for sachets, stickpacks or hard capsules).

It was an object of the present invention to provide an excipient with satisfying or improved flowability and/or compressibility and/or tableting capacity, and/or disintegration time.

It was an object of the present invention to provide a direct compression excipient.

It was an object of the present invention to provide an excipient that has the other required properties for this kind of excipient, for example in terms of granulometry, purity, safety.

### Presentation of the Invention

The inventor solved the above-mentioned problem(s) by providing granules of microcrystalline mannitol and of granular starch having a water activity equal to or lower than 0.3, said water activity being measured at 25 °C.

As evidenced in Example sections 1.2 and 2.2, FIGS. 2 and 10, the granules according to the disclosure have low water activity, lower than 0.3. When the granules were used for formulating probiotics, the water activity of the resultant formulations increased slowly, both in standard conditions (fridge or room temperature) or harsher conditions (30 °C at 65% relative humidity) (FIGS. 4, 5, 7, 13, 14, 16). In standard storage conditions (25 °C at 60% relative humidity), the water activity barely increased in powdery formulas packaged into aluminum foil bags, and was maintained in tablet formulas packaged into aluminum blisters (FIG. 13, 14, 16).

Without being bound by any theory, the inventors believe that this is due to the combined action of mannitol and granular starch. When the formula is prepared, the starch likely sequesters the water contained in the overall formula. Then, the starch and mannitol work together to prevent the moisture from reaching the probiotic: while mannitol can repel water and prevent additional water uptake due to its non-hygroscopic nature, the starch acts locally as a “sponge”, by sequestering the water contained in the micro-environment of the probiotic. Because granular starch is insoluble, the water remains trapped. When looking at the water activity of the formulations in FIGS. 4, 5, 7, 13, 14 and 16, it can be seen that after 3-month storage, the water activity of said formulations has not reached an equilibrium: the measured water activity is always lower than 0.60-0.65, i.e., lower than the equilibrium at 60%-65% relative humidity during storage.

Both granular starch and mannitol are essential. If mannitol alone is used as the excipient, the water is continuously repelled at the surface of the mannitol particles. As a result, the surface of mannitol particles is covered by free water which is directly impairing cell viability. If starch alone is used, all the surface of starch is adsorbing and consequently attracting water creating a flux of water, so it saturates more quickly, and water activity increases more rapidly.

The inventors also believe that the fact that mannitol and granular starch are co-processed is important because it maintains both components in the same micro-environments. The importance of having mannitol and granular starch present in a same granule was confirmed in later experiments, as shown in Example section 3.

As evidenced in Example section 2.4, FIGS. 18 and 19, and section 3.2, FIG. 24, the granules according to the disclosure allow formulating probiotics with improved stability. While the inventors were looking at an excipient that is not “too destabilizing”, they in fact developed an excipient that has a protective effect. The cell viability is improved when formulated into a powdery composition and is almost not impaired when formulated into tablets.

Furthermore, because the granules according to the disclosure have such a unique behavior with respect to water, they may be useful for formulating moisture-sensitive ingredients other than probiotics.

The granules according to the disclosure are particularly useful for making chewable tablets. Their tableting capacity is high enough for the tablets prepared therefrom to retain their integrity upon storage and transportation. At the same time, their tableting capacity is low enough for the tablets to be chewable. This is particularly advantageous for use in patients having difficulties to swallow, for example for pediatric or geriatric patients.

Granules having a mannitol to granular starch dry weight ratio greater than 65:35 and lower than 85:15, in particular greater than 75:25 and lower than 85:15, were found to be preferred for making tablets, because they allow to obtain tablets (i) of satisfying hardness, and also (ii) in which the cell viability is not too impaired by the compression. Granules having a mannitol to granular starch dry weight ratio of at least 75:25, in particular of at least 80:20, were found to be even more suitable.

Finally, the granules according to the disclosure exhibit excellent flowability, taste, and organoleptic properties.

The inventors also developed a useful process for making such granules. This process can be carried out at industrial scale. It has a good productivity, and can be carried out in a continuous way.

Co-processed excipients of microcrystalline mannitol and granular starch have already been described in previous patents in the name of the Applicant (U.S. Pat. Nos. 11,364,204 and 9,839,610). However, these co-processed excipients had a water activity of 0.5, i.e., higher than the water activity of the granules according to the disclosure. Furthermore, both documents never considered using such excipient for formulating moisture-sensitive ingredients, as these products were developed for a completely different purpose.

### BRIEF DESCRIPTION OF THE INVENTION

The invention first related to granules of microcrystalline mannitol and of granular starch having a water activity equal to or lower than 0.3, said water activity being measured at 25 °C.

Preferably, the granules according to the disclosure have a water activity equal to or lower than 0.2. Preferably, in the granules according to the disclosure, the dry weight ratio of mannitol to granular starch is equal to or higher than 50:50. Preferably, the granules according to the disclosure have a moisture content equal to or lower than 5.0% by weight. Preferably, the granules according to the disclosure have a bulk density equal to or higher than 350 g/L. Preferably, the granules according to the disclosure have a tapped density equal to or higher than 400 g/L. Preferably, the granules according to the disclosure have a specific surface area equal to or higher than 0.30 m²/g. Preferably, in the granules according to the disclosure, the mannitol comprises α- and -β polymorphs. Preferably, the granules according to the disclosure have a volume mean diameter equal to or higher than 90 μm, and equal to or lower than 400 μm.

The invention also relates to a process for making the granules according to the disclosure comprising a step (a) of agglomerating microcrystalline mannitol and granular starch by spraying and drying solubilized mannitol onto granular starch, a step (b) of drying the granules obtained in step (a), a step (c) of cooling the granules obtained in step (b), wherein steps (b) and (c) are performed using respectively a drying and a cooling air, said drying and cooling air both having a water content equal to or lower than 3.0 g per kg of dry air.

The invention also relates to granules of mannitol and granular starch obtained or obtainable by the process according to the disclosure.

The invention also relates to a product comprising the granules according to the disclosure, and another ingredient.

Preferably, said other ingredient is a moisture-sensitive active ingredient. Preferably, said other ingredient is a probiotic. Preferably, said product is a powdery composition, a tablet or a chewable tablet.

The invention also related to the use of the granules according to the disclosure for formulating or stabilizing a moisture-sensitive active ingredient.

### BRIEF DESCRIPTION OF DRAWINGS

Other features, details and advantages will be shown in the following detailed description and on the figures, on which:

FIG. 1 is a scheme of a device suitable for performing the process according to the disclosure.

FIG. 2 is a table showing the results of characterization of granules according to the disclosure.

FIG. 3 is a graph showing the tableting capacity of granules according to the disclosure.

FIG. 4 is a bar chart showing the water activity and moisture content of powdery compositions comprising granules according to the disclosure and a probiotic, upon 3-month storage at 30 °C/65% RH.

FIG. 5 is a bar chart showing the water activity and moisture content of tablets comprising granules according to the disclosure and a probiotic, upon 3-month storage at 30 °C/65% RH, said tablets being obtained using 15 kN compression force.

FIG. 6 is a bar chart showing the hardness and disintegration time of tablets comprising granules according to the disclosure and a probiotic, upon 3-month storage at 30 °C/65% RH, said tablets being obtained using 15 kN compression force.

FIG. 7 is a bar chart showing the water activity and moisture content of tablets comprising granules according to the disclosure and a probiotic, upon 3-month storage at 30 °C/65% RH, said tablets being obtained using 20 kN compression force.

FIG. 8 is a bar chart showing the hardness and disintegration time of tablets comprising granules according to the disclosure and a probiotic, upon 3-month storage at 30 °C/65% RH, said tablets being obtained using 20 kN compression force.

FIG. 9 are graphs showing cell viability of formulations (tablets and powdery compositions) comprising granules according to the disclosure. FIG. 9(a) is a bar chart showing cell viability, after compression, of tablets comprising granules according to the disclosure and a probiotic. FIG. 9(b) are graphs showing cell viability and water activity of formulations (tablets and powdery compositions) comprising granules according to the disclosure and a probiotic, upon 2-weeks storage at 30 °C/65% RH.

FIG. 10 is a table showing the results of characterization of granules according to the disclosure.

FIG. 11 is a graph showing the tableting capacity of granules according to the disclosure.

FIG. 12 are SEM pictures of granules according to the disclosure.

FIG. 13 is a bar chart showing the water activity and moisture content of powdery compositions comprising granules according to the disclosure and a probiotic, upon 3-month storage at 25 °C/60% RH or upon 3-month storage in the fridge.

FIG. 14 is a bar chart showing the water activity and moisture content of tablets comprising granules according to the disclosure and a probiotic, upon 3-month storage at 25 °C/60% RH or upon 3-month storage in the fridge, said tablets being obtained using 12.5 kN compression force.

FIG. 15 is a bar chart showing the hardness and disintegration time of tablets comprising granules according to the disclosure and a probiotic, upon 3-month storage at 25 °C/60% RH or upon 3-month storage in the fridge, said tablets being obtained using 12.5 kN compression force.

FIG. 16 is a bar chart showing the water activity and moisture content of tablets comprising granules according to the disclosure and a probiotic, upon 3-month storage at 25 °C/60% RH or upon 3-month storage in the fridge, said tablets being obtained using 16.5 kN compression force.

FIG. 17 is a bar chart showing the hardness and disintegration time of tablets comprising granules according to the disclosure and a probiotic, upon 3-month storage at 25 °C/60% RH or upon 3-month storage in the fridge, said tablets being obtained using 16.5 kN compression force.

FIG. 18 is a graph showing cell viability of formulations (tablets and powdery compositions) comprising granules according to the disclosure and a probiotic, upon 3-month storage at 25 °C/60% RH or upon 3-month storage in the fridge.

FIG. 19 represents four graphs showing cell viability and water activity of formulations comprising a probiotic (Lb. rhamnosus) either alone or in combination with granules according to the disclosure either in the form of a powdery composition or a tablet (12.5 kN or 16.5 kN compression force), upon 3-month storage at 25 °C/60% RH.

FIG. 20 represents 2 graphs showing the water activity of probiotic tablets (according to the disclosure or comparative), upon 3-month storage at 25 °C/60% RH or upon 3-month storage in the fridge, said tablets having a thickness of 6.9 mm.

FIG. 21 represents 2 graphs showing the moisture content of probiotic tablets (according to the disclosure or comparative), upon 3-month storage at 25 °C/60% RH or upon 3-month storage in the fridge, said tablets having a thickness of 6.9 mm.

FIG. 22 represents 2 graphs showing the hardness of probiotic tablets (according to the disclosure or comparative), upon 3-month storage at 25 °C/60% RH or upon 3-month storage in the fridge, said tablets having a thickness of 6.9 mm.

FIG. 23 represents 2 graphs showing the disintegration time of probiotic tablets (according to the disclosure or comparative), upon 3-month storage at 25 °C/60% RH or upon 3-month storage in the fridge, said tablets having a thickness of 6.9 mm.

FIG. 24 represents 2 graphs showing the cell viability of probiotic tablets (according to the disclosure or comparative), upon 3-month storage at 25 °C/60% RH or upon 3-month storage in the fridge, said tablets having a thickness of 6.9 mm.

### DESCRIPTION OF EMBODIMENTS

Figures and the following detailed description contain, essentially, some exact elements. They can be used to enhance understanding the invention and, also, to define the invention if necessary.

### Granules of Mannitol and Granular Starch

The invention first relates to granules of microcrystalline mannitol and of granular starch having a water activity equal to or lower than 0.3, said water activity being measured at 25 °C.

The expression “granules of mannitol and granular starch” classically refers to particles that, when observed by scanning electron microscopy at a magnification for example of ×200, have variable non spherical shape, and irregular surface.

In general, at a magnification of ×2500, fine particles of small agglomerated mannitol crystals can be seen at the surface of the granules.

The expression “microcrystalline” typically refers to a structure that, when observed by scanning electron microscopy at a magnification for example of ×2500, essentially presents microcrystals at its surface, and rarely crystals of higher size. A microcrystal might be defined as a crystal whose sum of length, width and thickness is lower than 25 μm. The microcrystals may have very different shapes, from rounded to elongated shapes.

Preferably, the mannitol of the granules according to the disclosure comprises both α- and β-crystalline form. The crystalline polymorphism of mannitol (crystalline forms and proportions) can be determined by those skilled in the art by infrared spectrometry or by diffraction X-rays on powder, preferably by diffraction of X-rays on powder. It can for example be determined according to the method described in the Examples section 1.2. Preferably, the mannitol of the granules according to the disclosure comprises less than 10% of δ-crystalline form, preferably, less than 5%, preferably less than 1%, preferably 0%. Still preferably, the mannitol of the granules according to the disclosure does not comprise δ-crystalline form, i.e., is composed of α- and β-crystalline forms only. Typically, the % ratio of α-form to β-form is from 5:95 to 95:5, preferably from 10:90 to 90:10, preferably from 15:85 to 85:15.

The expression “granular starch” classically refers to a starch whose granular structure is essentially retained. Granular starch typically has a structure organized as alternations of crystalline, semi-crystalline and amorphous layers. This organization is generally characterized by the observation of birefringence patterns under exposure to polarized light, also known as “Maltese crosses”. Granular starch is insoluble in water at room temperature (20-25 °C).

The granular starch according to the disclosure may be from any botanical origin. It is preferably an extra white starch, preferably an extra white corn starch.

It may be chemically, enzymatically, or physically modified, as long as its granular structure is retained. However, the granular starch according to the disclosure is preferably not modified, i.e., it is preferably native granular starch.

In general, the granular morphology starch can also be seen. Said granular starch being insoluble in water, it can be recovered by dissolving the granules according to the disclosure in water at room temperature.

The granules according to the disclosure can typically be obtained by agglomerating microcrystalline mannitol and granular starch. Thus, the granules according to the disclosure comprise or consist of co-processed (or “co-agglomerated”) microcrystalline mannitol and granular starch. They may be obtained by spraying and drying a liquid comprising solubilized mannitol and granular starch into a fluid bed. They may be obtained by spraying and drying a mannitol solution into a fluid bed containing granular starch, said granular starch being introduced into the fluid bed in the powdery state. During the drying phase, the liquid containing the solubilized mannitol evaporates, thus leading to the formation of microcrystals of mannitol that will be agglomerated with granular starch.

The agglomeration might be obtained by multi-stage spray-drying, or by fluid bed granulation. Typically, these processes include the recycling of fine particles.

In this respect, the inventors have observed that the granules of the disclosure prepared from a process including a spray-drying step conducted in a fluid bed, displayed advantageous features, notably a higher volume mean diameter, and an improved flowability as compared to those obtained from the same process including a spray-drying but not carried out in a fluid bed. Accordingly, the granules prepared from a process including a spray-drying step conducted in a fluid bed can be suitably used in the equipment for example used for tableting, or for filing sachet or stickpacks or hard capsules.

The granules according to the disclosure have a water activity equal to or lower than 0.30, preferably lower than 0.30, preferably equal to or lower than 0.20, preferably lower than 0.20. It is preferably equal to or higher than 0.10.

It is reminded that the expression “water activity”, also referred to as “Aw”, is the relative availability of water in a substance. The term “water activity” is typically defined as the vapor pressure of water in a sample divided by the vapor pressure of pure water at the same temperature. Pure distilled water has a water activity of exactly one. Water activity is temperature dependent. That is, water activity changes as the temperature changes. In the present disclosure, water activity is measured at 25 °C.

The water activity of a product can be determined by measuring the relative humidity of the air surrounding the sample at equilibrium. Accordingly, measurement of water activity in a sample is typically carried out in an enclosed (usually insulated) space where this equilibrium can take place. At equilibrium, the water activity of the sample and the relative humidity of the air are equal, and therefore a measurement of the equilibrium relative humidity (ERH) of the air in the chamber provides a measure of the water activity of the sample. At least two different types of water activity instruments are commercially available. One type of water activity instruments uses chilled-mirror dew point technology (e.g., AquaLab® water activity meters available from Decagon Devices, Inc.) while others measure relative humidity with sensors that change electrical resistance or capacitance (e.g., water activity meters available from Rotronic®).

This water activity can be determined by the person skilled in the art for example by capacitance hygrometers and dew point hygrometers, preferably by dew point hygrometers, for example according to the method described herein after in the Example section 1.2.

Preferably, the mannitol to granular starch dry weight ratio of the granules according to the disclosure is equal to or higher than 50:50, preferably equal to or higher than 55:45, preferably equal to or higher than 60:40, preferably equal to or higher than 65:35, more preferably higher than 65:35, preferably equal to or higher than 70:30, preferably equal to or higher than 75:25, more preferably higher than 75:25. It is preferably equal to or lower than 95:5, preferably equal to or lower than 90:10, preferably equal to or lower than 85:15, more preferably lower than 85:15. It is for example equal to about 80:20.

It is understood that this weight ratio refers to the dry weights of mannitol and granular starch used for making the granules. This dry weight ratio might differ a little from the amounts of mannitol and granular starch measured in the final granules. For example, and as it is apparent from the example herein after, for a dry weight ratio of mannitol/granular starch of 80:20, it can be seen that mannitol contents of from 75 to 84% were measured; this percentage being the percentage by weight of mannitol with respect to the total weight of granules.

This amount of mannitol might be determined by the person skilled in the art by high performance liquid chromatography on ion-exchange resin in calcium form, the quantification being performed using the external calibration method, for example according to the method described herein after in the Example section 1.2. Preferably, this measured amount of mannitol of the granules according to the disclosure is equal to or higher than 50% by weight, preferably equal to or higher than 55%, preferably equal to or higher than 60%, preferably equal to or higher than 65%, more preferably higher than 66%, preferably equal to or higher than 70%, more preferably higher than 70%, preferably equal to or higher than 75%. It is preferably equal to or lower than 95% by weight, preferably equal to or lower than 90%, preferably equal to or lower than 85%. It is for example equal to about 75% to equal to about 85%.

Preferably, the granules according to the disclosure have a moisture content (also referred to as “loss on drying”) equal to or lower than 5.0% by weight, preferably equal to or lower than 4.0%, preferably equal to or lower than 3.0%, preferably equal to or lower than 1.4%, preferably equal to or lower than 2.0%, preferably equal to or lower than 1.5%, preferably equal to or lower than 1.3%, preferably equal to or lower than 1.2%. It is in general higher than 0.1%, even higher than 0.2%. This moisture content can be determined by the person skilled in the art by halogen moisture analyzer or, most preferably, by drying of the test portion at a temperature ranging from 130 to 133 °C under air pressure, for 1h30, in an electrically-heated oven, for example according to the method described herein after in the Example section 1.2.

Preferably, the granules according to the disclosure have a bulk density equal to or higher than 350 g/L, preferably equal to or higher than 400 g/L, preferably equal to or higher than 450 g/L. It is in general equal to or lower than 750 g/L, even equal to or lower than 700 g/L, even equal to or lower than 650 g/L.

This bulk density might be determined by the person skilled in the art by the following method: flowing of a sample through a determined funnel into a test tube of known volume. Measurement of the mass, at a specified temperature, of a volume of the product in a test tube and determination of the volume of the test tube by measuring the mass of an equal volume of water at the same temperature. Calculation of the specific gravity of the product by dividing the mass of the product by the volume of the test tube. It is for example determined according to the method described herein after in the Example section 1.2.

Preferably, the granules according to the disclosure have a tapped density equal to or higher than 400 g/L, preferably equal to or higher than 450 g/L, preferably equal to or higher than 500 g/L. It is in general equal to or lower than 800 g/L, even equal to or lower than 750 g/L.

This tapped density might be determined by the person skilled in the art by the following method: Measurement of the mass of a volume of the product after compaction and of an equal volume of distilled water at the same temperature. Calculation of the specific gravity by dividing the mass of the volume of the product by the volume of the product itself. It is for example determined according to the method described herein after in the Example section 1.2.

The Hausner ratio can be calculated from those densities as follows:

$$Hausner ratio = \frac{Tapped density}{Bulk density} \qquad [ Math . 1 ]$$

The flow character can be determined from this Hausner ratio, according to the following Table 1:

TABLE 1

| Hausner ratio | Flow character |
|---|---|
| 1.00 to 1.11 | Excellent |
| 1.12 to 1.18 | Good |
| 1.19 to 1.25 | Fair |
| 1.26 to 1.34 | Passable |
| 1.35 to 1.45 | Poor |
| 1.46 to 1.59 | Very poor |
| ≥1.60 | Very, very poor |

Preferably, the granules according to the disclosure have good or excellent flow character (i.e., a Hausner ratio of from 1.00 to 1.18), preferably excellent flow character (i.e., a Hausner ratio of from 1.00 to 1.11).

Preferably, the granules according to the disclosure have a specific surface area equal to or higher than 0.30 m²/g, preferably equal to or higher than 0.40 m²/g, preferably equal to or higher than 0.50 m²/g, preferably equal to or higher than 0.80 m²/g, preferably equal to or higher than 1.00 m²/g, preferably equal to or higher than 1.20 m²/g, preferably equal to or higher than 1.40 m²/g, preferably equal to or higher than 1.50 m²/g. It is in general equal to or lower than 2.50 m²/g, even equal to or lower than 2.00 m²/g. This specific surface area can be determined by the person skilled in the art for example by using the BET method, for example according to the method described herein after in the Examples section 1.2.

Preferably, the volume mean diameter of the granules according to the disclosure is equal to or higher than 90 μm, and equal to or lower than 400 μm. It is preferably equal to or higher than 100 μm, preferably equal to or higher than 110 μm. It is preferably equal to or lower than 350 μm, preferably equal to or lower than 300 μm, preferably equal to or lower than 250 μm, preferably equal to or lower than 200 μm, preferably equal to or lower than 150 μm.

Preferably, the granules according to the disclosure have a D10 in volume equal to or lower than 100 μm, preferably equal to or lower than 80 μm, preferably equal to or lower than 60 μm, preferably equal to or lower than 50 μm. It is in general equal to or higher than 10 μm, even equal to or higher than 20 μm, even equal to or higher than 30 μm, even equal to or higher than 40 μm.

Preferably, the granules according to the disclosure have a D50 in volume from 50 to 180 μm, preferably from 70 to 160 μm, preferably from 90 to 140 μm, preferably from 100 to 130 μm, preferably from 110 to 120 μm.

Preferably, the granules according to the disclosure have a D90 in volume equal to or lower than 250 μm, preferably equal to or lower than 230 μm, preferably equal to or lower than 210 μm, preferably equal to or lower than 200 μm. It is in general equal to or higher than 120 μm, even equal to or higher than 140 μm, even equal to or higher than 150 μm, even equal to or higher than 160 μm, even equal to or higher than 170 μm.

It is reminded that the D10, D50 and D90 values in volume are the sizes, in μm, for which 10%, 50% and 90% respectively of particles, in volume, have a lower granulometry.

The volume mean diameter and particle size distribution in volume can be determined by determining the particle size distribution by laser diffraction on powdered products, for example according to the method described herein after in the Example section 1.2.

The granules according to the disclosure can be characterized in that they are “granules for direct compression” or “directly compressible granules”. It is also conventionally referred to as a “direct compression excipient”. The granules according to the disclosure can thus be compressed directly, i.e., without any prior texturing treatment or physical transformation, such as for example a prior dry or wet granulation step. This typically means that the granules are capable of forming tablets of sufficient hardness, by direct compression, in the sole presence of an efficient amount of lubricant. This “efficient amount” is such that it effectively allows the formation of tablets, that is to say typically, that there is no sticking, binding, and that the ejection force of the tablet from the press is less than 1000 Newtons, on a production for example of 10 tablets. This efficient quantity of lubricant generally does not exceed 3% by weight, relative to the total weight of the powder to be compressed. It is reminded that “binding” corresponds to the sticking of part of the material to the matrix, said sticking persisting after ejection of the tablet. Binding is visible on the tablet: vertical stripes are present and correspond to the locations where the product stuck to the matrix.

This ability to form satisfying tablets can be determined, for example, by direct compression of a pulverulent composition consisting of the excipient to be tested and lubricant, for example magnesium stearate, so as to form round flat faced tablets with a diameter of 11.28 mm, and a weight of 400±5 mg. Tablets can be formed by means of a rotary press, or by means of a single-punch compaction simulator which simulates compression on an industrial rotary press, for example such as that used in the Example section 1.2. The speed of the press can be set at 25 rotations per minute. A pre-compression force of 1.0 to 1.3 kN can be applied. When using the compaction simulator (STYL'One Evolution, Medelpharm), simulated to FETTE 2090 (EU-B) rotary tablet press, this speed corresponds to 54,000 tablets per hour at industrial scale.

On the tablets thus obtained, hardness measurements are carried out by means of a tablet hardness tester, for example such as that used in the Examples section below. The hardness of the tablets prepared from the excipient to be tested in the sole presence of the lubricant, expressed in Newtons (N), designates what is commonly referred to as “tableting capacity” of the excipient.

According to a “test A” in the present disclosure, the tableting capacity of the excipient to be tested is evaluated as follows: round flat faced tablets with a diameter of 11.28 mm and a weight of about 400±5 mg tablets are prepared on a single-point development press simulating compression on an industrial rotary press, simulated at a speed of 25 rpm on a FETTE 2090 (EU-B) rotary press, corresponding to 54000 tablets/hour, using pre-compression force of 1.0 to 1.3 kN and a compression force of 3 to 15 kN. Then, the hardness of the tablets is measured. According to this test, the tableting capacity is the maximum hardness obtained in this range of compression forces, said tablets presenting no breaking, sticking or binding on a production for example of 3 to 10 tablets, preferably of 3 or 10 tablets.

According to this test A, the granules according to the disclosure preferably have a tableting capacity equal to or higher than 40 N, preferably equal to or higher than 50 N, more preferably higher than 50 N, preferably equal to or higher than 60 N, preferably equal to or higher than 70 N, more preferably higher than 70 N, preferably equal to or higher than 80 N, preferably equal to or higher than 90 N, preferably equal to or higher than 100 N, preferably equal to or higher than 110 N, preferably equal to or higher than 120 N. It is preferably equal to or lower than 300 N, preferably equal to or lower than 250 N, preferably equal to or lower than 200 N, preferably equal to or lower than 150 N, preferably equal to or lower than 140 N, preferably equal to or lower than 130 N, preferably equal to or lower than 120 N.

The granules according to the disclosure are granules of microcrystalline mannitol and of granular starch, but they might include other ingredients, in small quantities, and as long as it does not interfere with the desired properties, notably in terms of efficacy and safety. Examples of such other ingredients are: binders, e.g., hydroxypropylmethylcellulose (HPMC), polyvinylpyrrolidone (PVP), carboxymethylcellulose (CMC), cellulose derivatives, acacia gum, gelatin, starch derivatives like maltodextrins, tragacanth gum; minerals; carbohydrates like sugars and sugar alcohols other than mannitol; food additives, colorants; pharmaceutical, nutraceutical, veterinary or cosmetic active ingredients; preservatives; stabilizers. Preferably, the amount of other ingredients in the granules according to the disclosure is lower than 15.0% (w/w), preferably lower than 10.0% (w/w), preferably lower than 5.0% (w/w), preferably lower than 2.0% (w/w), preferably lower than 1.0% (w/w), preferably lower than 0.5% (w/w).

Still preferably, the granules of mannitol and granular starch according to the disclosure are free of other ingredients. In the latter case, it means that the granules only consist in mannitol and granular starch, including their moisture and impurities.

### Process for Making the Granules of Mannitol and Granular Starch

The invention also relates to a process for making the granules according to the disclosure comprising a step (a) of agglomerating microcrystalline mannitol and granular starch by spraying and drying solubilized mannitol onto granular starch, a step (b) of drying the granules obtained in step (a), a step (c) of cooling the granules obtained in step (b), wherein steps (b) and (c) are performed using respectively a drying and a cooling air, said drying and cooling air both having a water content equal to or lower than 3.0 g per kg of dry air.

Preferably, the process according to the disclosure is a continuous process.

### Step (a)

In a first embodiment, the step (a) is performed by spraying a liquid, typically an aqueous composition, comprising solubilized mannitol and granular starch into a fluid bed. Granular starch being insoluble in water, the starch is suspended into the aqueous mannitol solution.

In a second more preferred embodiment, the step (a) is performed by spraying a mannitol solution, notably a mannitol aqueous solution, into a fluid bed while introducing powdery granular starch into said fluid bed.

The agglomeration may be performed by multi-stage spray-drying, or by fluid bed granulation. The step (a) will typically be performed into the chamber of a spray-dryer or of fluid bed granulator respectively. Both spraying and drying of step (a) will be performed in this unit.

Preferably, the step (a) is performed by multi-stage spray-drying.

It can be performed in a multi-stage spray dryer, (e.g., MSD®-type spray dryer), notably equipped with a high-pressure drying nozzle with recycling of the fine particles at the spray dryer top.

While the water content of the air of the drying step (b) must be carefully controlled, the water content of the drying air of step (a) doesn't need such control.

The temperature of the drying air of step a) can be easily adjusted by the person skilled in the art, depending on the equipment used. This temperature will be typically higher than 100 °C.

Preferably, the mannitol to granular starch dry weight ratio used for performing step (a) is as described before for the granules according to the disclosure. Thus, the mannitol to granular starch dry weight ratio used to prepare the liquid to be sprayed in step (a) may notably range from 50/50 to 95/5.

Preferably, the liquid or solution sprayed in step (a) comprises from 30 to 50% of solids, preferably from 35 to 45%, for example about 40%; said percentage being expressed in dry weight of solids with respect to the total weight of said liquid or solution.

### Steps (b) and (c)

In the process according to the disclosure, the drying and cooling airs of steps (b) and (c) have a water content equal to or lower than 3.0 g per kg of dry air. Preferably, the drying and cooling airs of steps (b) and (c) have a water content equal to or lower than 2.5 g per kg of dry air, preferably equal to or lower than 2.0 g per kg of dry air, preferably equal to or lower than 1.5 g per kg of dry air, preferably equal to or lower than 1.0 g per kg of dry air. It is in general equal to or higher than 0 g/kg of dry air, even equal to or higher than 0.5 g/kg of dry air. It is for example equal to about 0.6 g/kg of dry air.

The steps (b) and (c) of drying and cooling may be performed in a unit separated from the fluid bed granulator or spray-drying chamber, in particular when a continuous process is used.

Preferably, the drying step (b) is performed in a fluid bed, preferably selected from a static fluid bed and/or a vibrated fluid bed. It is still preferably performed in a static fluid bed and, preferably subsequently, in a vibrated fluid bed.

Preferably, the cooling step (c) is performed in a vibrated fluid bed, preferably in the same vibrated fluid bed as used for the drying step (b).

The temperatures of the drying and cooling airs of step (b) can be easily adjusted by the person skilled in the art, depending on the equipment used, and based on the detailed process(es) given in the Examples herein after.

For example, the temperature of the drying air of step (b) can be selected from 50 °C to 130 °C, preferably from 60 °C to 120 °C, preferably from 70 °C to 110 °C, for example from about 75 °C to about 100 °C. When a static fluid bed and a vibrated fluid bed are used for the drying step (b), the air temperature of the static fluid bed can be from 50 °C to 100 °C, preferably from 60 °C to 90 °C, preferably from 70 °C to 80 °C, for example of about 75 °C. When a static fluid bed and a vibrated fluid bed are used for the drying step (b), the air temperature of the vibrated fluid bed can be from 70 °C to 130 °C, preferably from 80 °C 120 °C, preferably from 90 °C to 110 °C, for example of about 100 °C.

The temperature of the cooling air of step (c) can be from 5 to 30 °C, preferably from 10 to 25 °C, for example of about 20 °C.

Preferably, the process includes the recycling of fine particles. They may be recycled in the powdery state or via solubilization into the mannitol liquid sprayed. It is preferably recycled into the powdery state. The step of recycling can be easily carried out by the person skilled in the art. The recycling may be performed by extracting fine particles at the top of the chamber. After being extracted, the fine particles may be then separated from the coarse particles eventually present by way of a cyclone, before being reintroduced into the chamber, for example at the top of the chamber.

When the granular starch is introduced into the powdery form, it can advantageously be introduced via the inlet used of the recycling of powdery fine particles.

Preferably, the process according to the disclosure further includes a step (d) of recovering the granules thus obtained.

Other process parameters can be easily adjusted by the person skilled in the art, depending on the equipment used, and based on the detailed process(es) given in the Examples herein after. Examples of such other process features are: the spraying pressure, the type and number of nozzles used, the flow rate for the sprayed solution or liquid of step (a), the flow rate of granular starch when the latter is introduced in the powdery form.

If the granules according to the disclosure include other ingredients, such other ingredients are typically introduced during step (a). They may be introduced via the liquid or solution sprayed, via an inlet for recycling the fine particles, via the inlet used for introducing granular starch, or via an additional inlet.

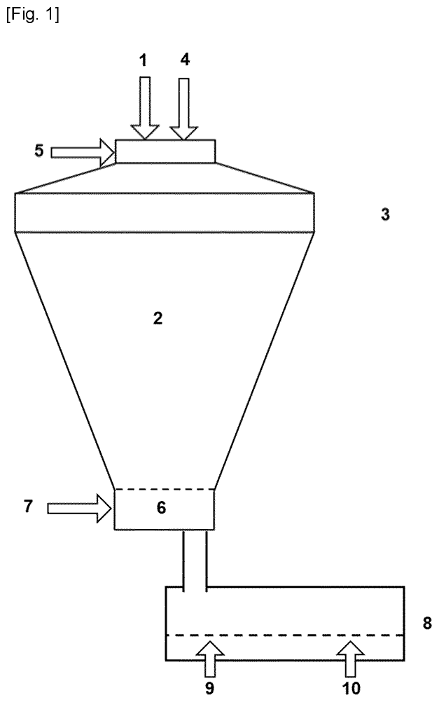

FIG. 1 is a scheme of a preferred device for performing the process according to the disclosure. In this equipment, a mannitol solution (1) is sprayed into the drying chamber (2) of a multi-stage spray-drier (3), via a spraying nozzle. Granular starch is introduced into the powdery state via the inlet (4) also used for recycling powdery fine particles. A drying air (5) is introduced into the chamber (2) for performing the drying of step (a). The step (b) of drying is performed into a static fluid bed (6), fed with a drying air (7) having a water content equal to or lower than 3.0 g per kg of dry air, and then into a vibrated fluid bed (8) fed with a drying air (9) having a water content equal to or lower than 3.0 g per kg of dry air. The step (c) of cooling is performed into the vibrated fluid bed (8) fed with a cooling air (10) having a water content equal to or lower than 3.0 g per kg of dry air. The fine particles are recycled via the inlet (4).

Once prepared, the granules of the disclosure are preferably stored under conditions enabling to maintain a water activity equal or lower to 0.3 over time, notably to preserve the granules from the moisture of the environment, particularly in view of their potential combination with a moisture sensitive ingredient. As an example, the granules may be stored at room temperature, i.e from 18 to 25 °C, or in the fridge. Preferably, the granules according to the disclosure are stored in aluminum foil packaging.

The invention also relates to granules of microcrystalline mannitol and granular starch obtained or obtainable by the process according to the disclosure.

### Products Comprising the Granules of Mannitol and Granular Starch

The invention also relates to a product comprising the granules according to the disclosure and another ingredient, preferably a moisture-sensitive active ingredient.

Preferably the product is selected from powdery compositions and/or tablets.

Preferably, the product comprises a moisture-sensitive active ingredient. The active ingredient according to the disclosure includes non-pharmaceutical and pharmaceutical agents. The expression “active ingredient” classically refers to any substance of pharmaceutical, veterinary, food, nutraceutical, or cosmetic interest. Preferably, the active ingredient according to the disclosure is a nutraceutical or pharmaceutical active ingredient. The active ingredient, in particular when it is a pharmaceutical active ingredient, may be chosen from so-called small molecules, but also from so-called “biopharmaceuticals”, as is the case for example of active substance based on or derived from proteins, nucleic acids (e.g., DNA or RNA), cells or viruses.

The expression “moisture-sensitive active ingredients” is well understood in the field. It typically refers to active ingredients that exhibit undesirable changes when exposed to moisture, which may come from various sources—excipients, manufacturing processes, environmental conditions etc. A common result of contact between a moisture-sensitive active ingredient and moisture is the formation of one or more different chemical species. The consequence could be a hydration of the drug molecule, resulting in a change in physical properties (e.g., different dissolution profile), or it could produce different compounds or degradants, leading to a loss of potency. For probiotics, it is known that moisture exposure needs to be limited to minimize decrease in cell viability due to detrimental chemical reactions.

In some instances, drug stability is evidenced by a slow rate of degradant compound formation, overtime. The period of time, during which a drug must remain stable, i.e., maintain its potency and/or impurity content in a formulation, varies according to commercial specifications set by the manufacturer. For example, a particular product might be required to maintain certain potency specifications for a period of six months, one year, two years, or some other time following manufacturing. The established shelf life of a product presumes maintenance in the original packaging, in specified temperature and humidity environments.

Non-limiting examples of moisture-sensitive active ingredients are probiotics, for example bacteria or yeasts, preferably from Lactobacillus or Bifidobacterium genus, drug molecules such as, dabigatran, enalapril, ranitidine, omeprazole, aspirin, ascorbic acid. It is preferably selected from probiotics, preferably from bacteria, more preferably from lactobacillus bacteria, e.g., Lb. rhamnosus.

Preferably, in the products according to the disclosure, the weight ratio of granules according to the disclosure:moisture-sensitive active ingredient is from 1:1 to 10:1, preferably from 1:1 to 8:1, preferably from 1:1 to 5:1, preferably from 1:1 to 4:1, preferably from 2:1 to 4:1. It is for example equal to about 3:1.

Preferably, the products according to the disclosure have a water activity equal to or lower than 0.50, preferably equal to or lower than 0.45, preferably equal to or lower than 0.40, preferably equal to or lower than 0.35, preferably equal to or lower than 0.30, preferably equal to or lower than 0.25, preferably equal to or lower than 0.20. It is preferably equal to or higher than 0.01, preferably equal to or higher than 0.05.

This water activity can be determined by the person skilled in the art for example by capacitance hygrometers and dew point hygrometers, preferably by dew point hygrometers, for example according to the method described herein after in the Example section 1.3.

Preferably, the products according to the disclosure have a moisture content (also referred to as “loss on drying”) equal to or lower than 5.0% by weight, preferably equal to or lower than 4.0%, preferably equal to or lower than 3.0%, preferably equal to or lower than 2.0%. It is in general higher than 0.5%, even higher than 1.0%. This moisture content can be determined by the person skilled in the art by halogen moisture analyzer or water oven 130 °C, most preferably halogen moisture analyzer, for example according to the method described herein after in the Example section 1.3.

In a first preferred embodiment, the product according to the disclosure is a tablet, preferably a chewable tablet. It is understood that some of the features of the granules (their diameter or densities for example), once tableted, might not be found in the final tablets. Therefore, alternatively, the tablets according to the disclosure might be defined by the fact that they are obtained or obtainable from a powdery composition comprising the granules according to the disclosure.

Preferably, the tablets according to the disclosure are biconvex caplets, preferably having a width of 9.5 mm and a length of 19 mm.

Preferably, the tablets according to the disclosure have a weight of about 1 g.

Preferably, tablets according to the disclosure have a hardness equal to or higher than 40 N, preferably equal to or higher than 50 N, preferably equal to or higher than 60 N, preferably equal to or higher 70 N. Preferably, tablets according to the disclosure have a hardness equal to or lower than 250 N, preferably equal to or lower than 200 N, preferably equal to or lower than 150 N, preferably equal to or lower than 140 N, preferably equal to or lower than 130 N, preferably equal to or lower than 120 N. It is for example selected from 60 to 130 N, or from 70 to 120 N.

In a second preferred embodiment, the product according to the disclosure is a powdery composition. The powdery composition might be a powdery composition suitable for making another product (for example a premix for making a tablet). The powdery composition might be a filler for hard capsules, sachets or stickpacks.

In a first preferred embodiments, the granules or products according to the disclosures are stored in the fridge i.e., at a temperature of from 4 to 8 °C.

In a second preferred embodiments, the granules or products according to the disclosure are stored at temperature of 15 to 30 °C, preferably of from 20 to 30 °C, still preferably of from 20 to 25 °C, still preferably of about 25 °C, at the relative humidity equal to or lower than 60%. This relative humidity is preferably as low as possible, for example of about 20-30%.

Preferably, the products according to the disclosure are in aluminium foil packaging. The products according to the disclosure may be packaged in bags, in bottles, in blisters, in sachets or in stickpacks. Preferably, the tablets according to the disclosure are packaged in bags, bottles, or blisters, still preferably in blisters, preferably in aluminum blisters. Preferably, the powdery compositions according to the disclosure are packaged in bags, sachets or stickpacks, preferably in aluminium sachets or aluminium stickpacks. The powdery compositions may be used as a filler for hard capsules. In that case, the hard capsules are preferably packaged in bags, bottles, or blisters, still preferably in blisters, preferably in aluminum blisters.

The products according to the disclosure may be packaged together with a desiccant pack, for instance a pack containing silica beads. This might be interesting in particular to protect the product from the moisture of the environment caused by multiple openings of the package by the user. Such desiccant pack can typically be added into bottles.

### Uses of the Granules of Microcrystalline Mannitol and Granular Starch

The invention also covers the use of the granules according to the disclosure, for formulating or stabilizing a moisture-sensitive active ingredient.

Preferably, the granules are as described before. Preferably, the moisture-sensitive active ingredient is as described before. In particular, it is preferably selected from bacteria or yeasts, preferably from Lactobacillus or Bifidobacterium genus, or from drug molecules such as, dabigatran, enalapril, ranitidine, omeprazole, aspirin, ascorbic acid. It is preferably selected from probiotics, preferably from bacteria, more preferably from lactobacillus bacteria, e.g., Lb. rhamnosus.

Preferably, the granules are used for formulating a product which is preferably as described before. In particular, said product is preferably selected from tablets or powdery compositions.

Preferably, the granules are used as a filler and/or as a binder and/or as a direct compression excipient.

This ability of stabilizing moisture-sensitive active ingredients can be determined for example by subjecting a formulation consisting in the granules, the moisture-sensitive active ingredient and 1 to 3% by weight of lubricant to storage at a temperature of 30 °C and a relative humidity of 65%, for a given duration, for example for at least 3 months, or from 3 to 12 months, or from 3 to 6 months, for example for 3 months. It may also be determined at a temperature of 25 °C and a relative humidity of 60%. If the loss of activity is lower in the formulation as compared to the active ingredient alone, it can be considered that the active ingredient is stabilized. For probiotics, this can be done by measuring cell viability, for example according to the method given in the Example section 1.4.

Preferably, the granules and products comprising them according to the disclosure, in particular the tablets according to the disclosure, are for individuals that have difficulties to swallow, and/or for children and/or for the elderly and/or for pediatric patients and/or for geriatric patients and/or for patients suffering from dysphagia.

In the instant disclosure, the amounts of ingredients are generally expressed in percentages by weight. Unless otherwise specified these weights are amounts of ingredients as such, in their powdery or oily form. Powdery ingredients generally include small amount of water (also referred to as % moisture or as “loss on drying”) and/or small amounts of impurities.

Other characteristics and advantages of the present invention will emerge clearly on reading the examples given hereinafter, which illustrate the invention without however limiting it.

### EXAMPLES

### 1. Prototypes of Granules of Mannitol and Starch

### 1.1. Preparation of Granules of Mannitol and Starch in Batch Mode, Using a Fluid Air Bed Granulator

Different compositions consisting of mannitol and of granular starch at dry weight ratios respectively of 85:15, 80:20, 75:25 and 65:35 were prepared by agglomeration and drying according to the disclosure.

A solution of mannitol and of granular starch at the desired solids content was prepared by dissolving mannitol (PEARLITOL® 50C, ROQUETTE) in demineralized water at 55 °C and by suspending extra white corn starch. Stirring was performed so as to obtain a fluid and homogeneous solution devoid of lumps.

The operating conditions for the manufacture of these granules in the batch fluid air bed granulator of AGT 150 type sold by Glatt appears in the following Table 2. The spray nozzle was in the “bottom spray” position.

TABLE 2
step (a)

| | G-MS- | G-MS- | G-MS- | G-MS- |
|---|---|---|---|---|
| | lowAw- | lowAw- | lowAw- | lowAw- |
| | 65/ | 75/ | 80/ | 85/ |
| | 35_#1 | 25_#1 | 20_#1 | 15_#1 |
| Mannitol to granular starch | 65:35 | 75:25 | 80:20 | 85:15 |
| dry weight ratio | | | | |
| Total solid content of the | 35 | 40 | 35 | 35 |
| liquid sprayed (%) | | | | |
| Feed flow rate (g/h) | 3750 | 3750 | 4100 | 4000 |
| Air flow rate (Nm³/h) | 110 | 110 | 110 | 110 |
| Inlet air (°C) temperature | 125 | 125 | 120 | 125 |
| Product bed temperature | 60 | 60 | 54 | 58 |
| (°C) | | | | |
| Nozzle spray pressure | 1.2 | 1.0 | 1.0 | 1.0 |
| (bar) | | | | |

For performing step (b) of drying, granules obtained where put in a ventilated oven and dried for 48 h at a temperature of 80 °C, using air with controlled humidity (lower than 3.0 g per kg of dry air). For performing step (c) of drying, the granules were cooled to room temperature, using air with controlled humidity (lower than 3.0 g per kg of dry air) for 24 h.

### 1.2. Granules' Characterization

The granules thus obtained were characterized according to the following methods.

1. Water activity (Aw). Aw was measured using the device Waterlab, STEROGLASS®. Analysis was carried out at 25 °C. Analysis was done based on equipment recommendation (WATERLAB_MU_ENG_Rev2_2020).

2. Mannitol content. The mannitol content was determined by high performance liquid chromatography on ion-exchange resin in calcium form. Quantification was performed using the external calibration method. The following material and parameters were used: Eluent: water; Flow rate: 0.5 ml/min; Column temperature: 85 °C; Volume injected: 20 μL; A column of ion-exchange resin in calcium form: BIORAD HPX87C in Ca++ form (300×7.8 mm) (ref. 125-0095 for example); Detector: differential refractometer.

3. Moisture content. Moisture content was determined by drying of the test portion, at a temperature ranging from 130 to 133 °C under air pressure, for 1.5 hours, in an electrically-heated oven, according to the following protocol: introduce a test portion of about 5 g, accurately weighed, of the test sample. Disperse it in a uniform thin layer onto the bottom of a capsule. Place the uncovered capsule in the oven leaving the lid nearby, for 1h30, from the moment when the oven regulates again in the defined range of temperature. Do not open the oven during drying.

4. Bulk and Tapped densities, Hausner ratio. The bulk density was determined by the following method: flowing of a sample through a determined funnel into a test tube of known volume. Measurement of the mass, at a specified temperature, of a volume of the product in a test tube and determination of the volume of the test tube by measuring the mass of an equal volume of water at the same temperature. Calculation of the bulk density of the product by dividing the mass of the product by the volume of the test tube. The following material and parameters were used: Temperature: 20 °C; Stainless funnel: upper diameter 12 cm, lower diameter 12 mm, cone height 9 cm, tube length 2 cm. This funnel was placed on a stand so that the distance between the funnel and the test tube be 10 cm.

Tapped density was determined the following method: Measurement of the mass of a volume of the product after compaction and of an equal volume of distilled water at the same temperature. Calculation of the tapped density by dividing the mass of the volume of the product by the volume of the product itself. The protocol was the following: weigh 20 g powder in the test tube. Place the test tube on the tapped density tester (STAV 2003 tapped density tester) and turn on at 50 strokes. Read the volume in the test tube.

5. Specific surface area. Specific surface area of the powdery granules was measured based on the 3-point Brunauer, Emmett and Teller (BET) theory. The physical principal used to determine the specific surface area is based on low temperature gas adsorption. Apparatus—Beckman-Coulter SA3100.

6. Crystalline polymorphism. Crystalline polymorphism was determined by diffraction of X-rays on powder. The determination of the crystalline forms of mannitol as well as their quantifications were carried out using a powder X-ray diffraction spectrometer (Bruker) with a copper anode tube (wavelength: 1.54 Å). The analysis was performed continuously, from 5 to 60°, in reflection, with a rotating sample holder. The sample was compacted manually and deposited in a flat layer on the sample holder. The crystalline forms of mannitol were determined by comparing the positions of the diffraction lines of the sample against the alpha, beta and delta forms of mannitol using the fundamental parameters approach from the structure files available in the COD (Crystallography Open Database) and CSD (Cambridge Structural Database) databases. Ref: Alpha ref CSD1142501/Beta ref CSD1142500/Delta ref CSD 662815.

7. Volume mean diameter and particle size distribution in volume. Volume mean diameter and particle size distribution in volume were determined by laser diffraction on powdered products. Measurement of the diffraction of a laser beam by the particles of the sample to be analyzed. These particles diffract individually. The resulting diffraction light intensity is a function of the particle size. The computer processing of these light intensities gives access to particle size distributions. Equipment: LASER diffraction particle size meter HELOS KR Brand SYMPATEC, equipped with R5 and R7 lenses. RODOS M system associated with a 6 mm gun and a VIBRI system. Vacuum cleaner Nilfisk IBVI5.

8. Tabletinq capacity. The excipient to be tested was mixed with 1% magnesium stearate. Round flat faced tablets with a diameter of 11.28 mm, and a weight of about 400 mg were prepared on a single-punch compaction simulator (STYL'One Evolution, Medelpharm), simulated at a speed of 25 rpm on a FETTE 2090 (EU-B) rotary press, corresponding to 54000 tablets/hour, using pre-compression force of 1.0 to 1.3 kN. Different compression forces were tested, from about 3 to 20 kN. The hardness of the tablets thus obtained was measured with a tablet hardness tester (ERWEKA TBH 425, ERWEKA GmBH).

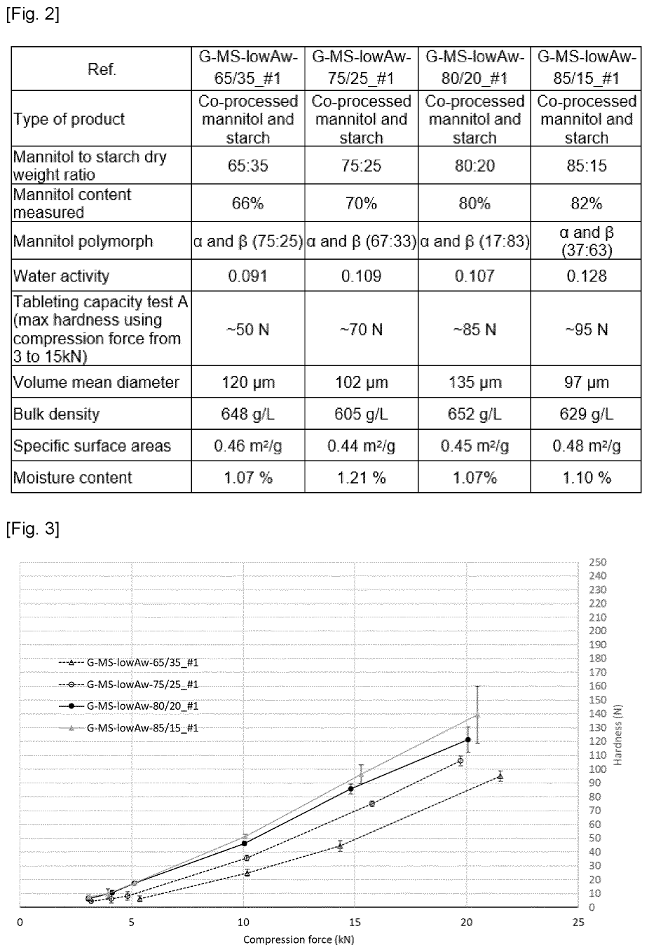

The results are presented in FIGS. 2 and 3.

The granules according to the disclosure all had low water activity, lower than 0.3. The water activity and tableting capacity increased with the mannitol to granular starch dry weight ratio (FIGS. 2 and 3).

The granules according to the disclosure have satisfying tableting capacity (FIG. 3). They are thus suitable for making tablets with sufficient hardness, i.e., that won't break during storage and transportation. On the other hand, the hardness is not too high, so the tablets can easily be chewed. The granules according to the disclosure are thus particularly suitable for making chewable tablets.

There are no ideal values of hardness for a tablet to be chewable, as it will depend on the size and shape of the tablet. However, in general, hardness is preferably of at least 60-70 N. Therefore, the granules having a mannitol to granular starch dry weight ratio greater than 65:35 are preferred for making tablets, due to their higher tableting capacity. Granules having a mannitol to granular starch dry weight ratio of at least 75:25 are even more preferred.

Granules according to the disclosure typically exhibit a volume mean diameter and size distribution that make them suitable for use in tableting or for filing for example hard capsules, sachets, or stickpacks. This allows them to flow properly into the devices used for making and packaging formulas comprising them. It also enables homogenous blending with most of active ingredients.

### 1.3. Stability of Formulations Comprising the Granules and a Probiotic

Lactobacillus rhamnosus was selected as the model strain because it is a well-known and well-researched probiotic strain, and is found in many commercial probiotic formulations for multiple age groups.

Two types of formulations were tested: tablets, and powdery compositions.

The formulations used after optimization was the following by weight: 1% magnesium stearate, 74.25% prototype, 24.75% Lb. rhamnosus. Same formulations were used for the tablets and the powdery compositions.

The tablets were made as follow: biconvex caplets with a length of 19 mm, a width of 9.5 mm and a weight of about 1 g were prepared on a single-punch compaction simulator (STYL'One Evolution, Medelpharm), simulated at a speed of 25 rpm on a FETTE 2090 (EU-B) rotary press, corresponding to 54000 tablets/hour, using pre-compression force of 1.0 to 1.3 kN, and compression forces of 15 kN or 20 kN (selected after optimization).

For the stability studies, the tablets and powdery compositions were packaged into aluminum foil bags having low headspace so as to minimize the quantity of air in contact with the granules.

The stability was evaluated by measuring the Aw and moisture content upon storage at 65% RH/30 °C, according to the following methods.

1. Water activity. The water activity was measured using a water activity meter (Aqualab 4TE Duo, Meter Group, USA) at 25 °C. Powdery composition samples were measured immediately after the package is opened with a sample weight of 1 g (±0.05 g) while the tablets (sample weight of 1 g (±0.05 g)) were crushed prior to analysis to reduce the equilibration time within the water activity chamber.

2. Moisture content. The Loss on Drying (LOD) was measured using a halogen moisture analyzer (Mettler Toledo, HC103) at a drying temperature of 105 °C. Powdery composition samples were measured immediately after the package is opened with a sample weight of 1 g (±0.05 g) while the tablets (sample weight of 1 g (±0.05 g)) were crushed prior to analysis.

For the tablets, the hardness and disintegration time upon storage were also assayed. Disintegration time was conducted according to the European Pharmacopoeia disintegration test for tablets and capsules (EP 5.0 2.9.1.) using a disintegration tester (PTZ Auto, Pharma Test Apparatebau AG, Germany).

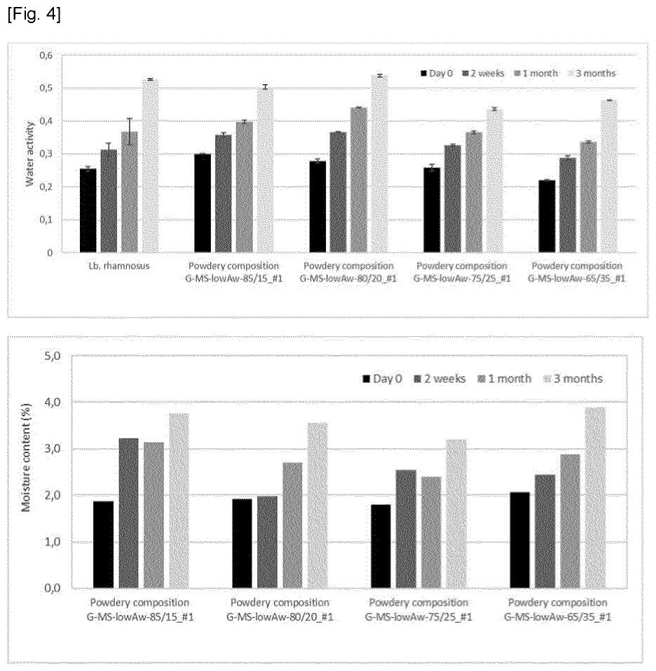

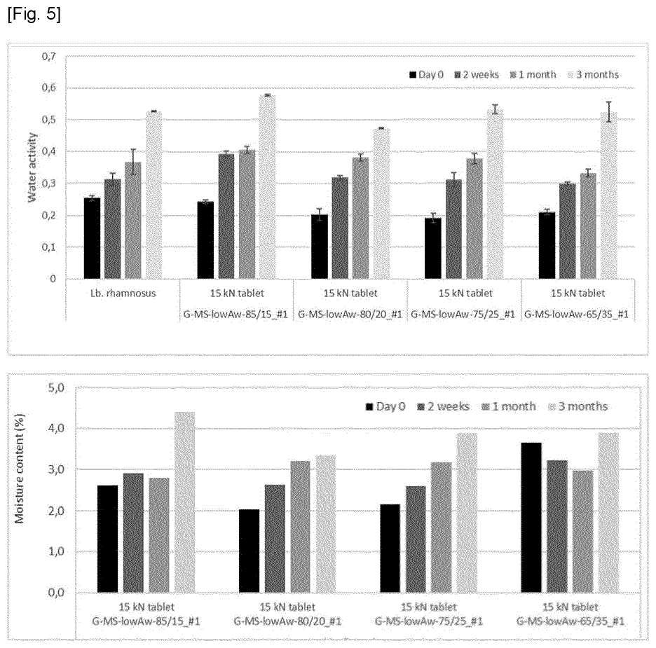

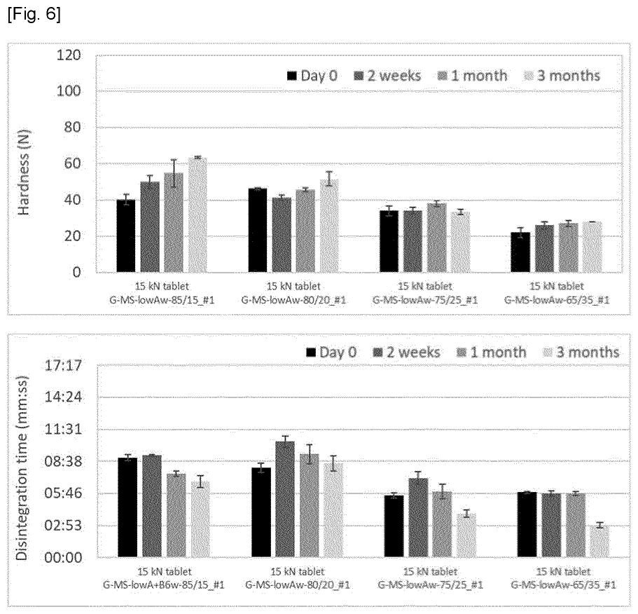

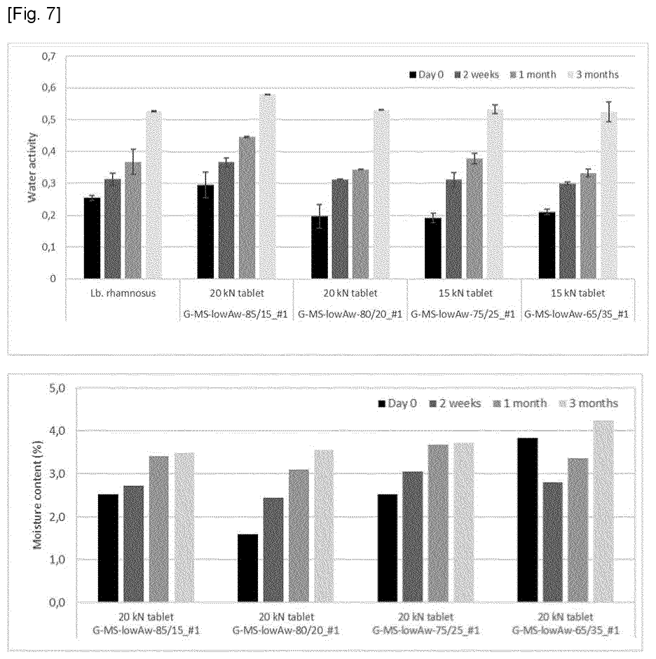

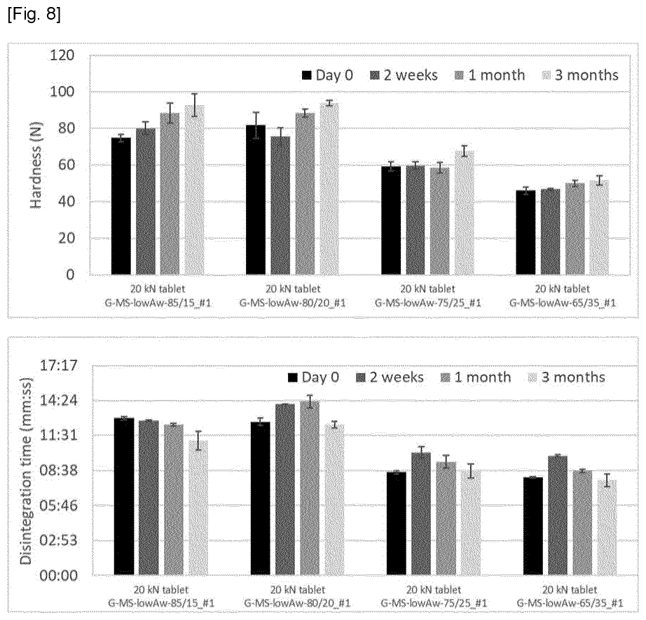

Results are presented in FIGS. 4 to 8.

These results show that the tablets and powdery compositions obtained from the granules according to the disclosure have good stability.

After 3 months of storage at 65% RH/30 °C, the water activity gain for Lb. rhamnosus was 0.27. For the powdery compositions and tablets, an increase in water activity was observed. The increase in water activity ranged from 0.18 to 0.26 for the powdery compositions, and from 0.26 to 0.34 for tablets. Generally, it can be observed that the incorporation of granules in the formulation did not result in an enhanced uptake of free water during storage, insofar as the final water activity after 3 months was comparable to that of Lb. rhamnosus alone. Thus, unexpectedly, these results show that the granules do not bring any additional free water to the formulation.

As a further unexpected effect, the data hereinafter comparing the cell viability of Lb. rhamnosus alone, on the one hand, and in admixture with the granules, on the other hand, surprisingly demonstrate that not only the cell viability is not negatively impaired over time by the granules, but to the contrary, the presence of the granules even enable to improve the cell viability over time and hence to protect Lb rhamnosus from moisture.

The hardness and disintegration time of the tablets are steady over the time, which is a sign of physical stability of the tablets.

Prototype including granules [G-MS-lowAw-85/15_#1] and [G-MS-lowAw-80/20_#1] consistently resulted in higher tablet hardness followed by [G-MS-lowAw-75/25_#1] and then [G-MS-lowAw-65/35_#1], which is consistent with the tableting capacity data showed in section 1.2. The disintegration time increased with the tablet hardness.

1.4. Cell Viability in Formulations with Probiotics

The inventors checked the impact of compression on cell viability. Cell viability was assayed with the 3M Petrifilm Lactic Acid Bacteria Count Plate method. Samples (equivalent to 1.0±0.1 g) were aseptically weighed out and added to 9 mL peptone water diluent (3M). The suspension was mixed with a Turbula blender (Turbula T2F, Glen Mills) for 5 mins and visually checked for homogeneity. If sample was not well dispersed, the suspension would be mixed for another 5 mins. The suspension was allowed to stand at room temperature for 25 mins after 5 mins of mixing, to make up a total of 30 mins for sample hydration. The total time for mixing and sample standing is 30 mins. If the mixing step took 10 mins, the standing time would be 20 mins accordingly. After a homogenous suspension was obtained, the suspension was diluted to achieve the theoretical concentration that would result in a count between 20 and 300 colonies. Prior to dilution and plating steps for each sample, the sample would be vortexed for 30 seconds. The appropriate diluted samples was plated on the 3M Petrifilm Lactic Acid Bacteria Count plate according to the manufacturer's instructions, and incubated for 48±3 hours at 28-37 °C.

The 3M Petrifilm Lactic Acid Bacteria Count Plate method was validated with ISO 15214 pour plate method, where no significant difference between the two test methods were observed based on the means of the sample replicates as determined by the paired t-test (p=0.43).

Tablets were prepared as in section 1.3.

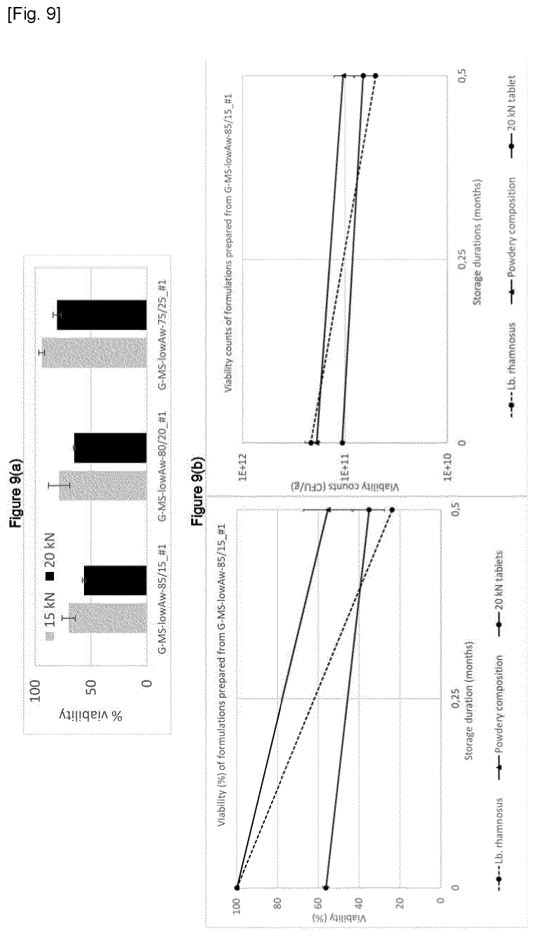

Results are presented in FIG. 9(a).

It can be observed that the impact of compression force on cell viability is secondary to that of the mannitol to granular starch ratio. Regardless of the mannitol to granular starch ratio, tablets compacted at 20 kN consistently had 13% lower cell viability compared to tablets compacted at 15 kN. The minimum cell viability was obtained with the granules having a mannitol to granular starch dry weight ratio of 85:15 using a compression force of 20 kN (56% cell viability). The maximum cell viability was obtained with the granules having a mannitol to granular starch dry weight ratio of 75:25 using a compression force of 15 kN (94% cell viability).

As a comparison, a commercial excipient for tableting of probiotics (made of different materials) showed a cell viability of 30-40% using a compression force of 15 kN, and of about 20% using a compression force of 20 kN.

These results also show that the higher the granular starch content, the higher the cell viability. With that respect, a mannitol to granular starch dry weight ratio lower than 85:15 is thus preferred when the granules are used for making tablets.

Cell viability was then evaluated on tablets and powdery compositions prepared as described in section 1.3, and stored at 30 °C/65% RH for two weeks. The granules having a mannitol to granular starch dry weight ratio of 85:15 was selected for this evaluation. Indeed, because these granules had the highest water activity and lowest cell viability after compression, the hypothesis was made that the other granules having higher starch content would perform at least equally, if not better. For the tablets, the 20 kN compression force was selected for similar reason i.e., because the use of this compression force resulted in the lowest cell viability.

Results are presented in FIG. 9(b).

After 2-week storage, both tablets and powdery formulations showed a higher cell viability, as compared to the probiotic alone. These results show the protective effect of the granules according to the disclosure. Even if the cell viability was impaired by tableting, this loss was rapidly balanced by the fact that the cell viability decreased much slower in the tablets as compared to the untableted probiotic.

### 2. Pilot Batches of Granules of Starch and Mannitol

For the scale-up, the mannitol to granular starch dry weight ratio of 80:20 was selected, as it was the preferred one identified in the previous set of experiments, for making chewable tablets. Indeed, granules having a mannitol to granular starch dry weight ratio greater than 75:25 and lower than 85:15 are the best compromise between Aw, tableting capacity, and cell viability.

### 2.1. Preparation of Granules of Starch and Mannitol in Continuous Mode, Using a Multi-Stage Spray-Drier (MSD)

Granules consisting of mannitol and granular starch at a dry weight ratio of 80:20, were prepared by multistage spray-drying. Use was made of crystalline mannitol, sold by the Applicant under the name PEARLITOL® 50C, exhibiting a laser volume mean diameter of approximately 50 μm and of “extra white” corn starch. A mannitol solution at the desired solids content was prepared by dissolving the crystalline mannitol in demineralized water at 80 °C. The granular starch was introduced in the powdery form in the system for recycling of the fine particles via a weight powder dosing device. The operating conditions for the manufacture of these granules in the MSD type spray-dryer sold by Niro with an evaporation capacity of 400 kg/h appear in the following Table 3. Fines particles are recycling at the top of the chamber.

TABLE 3

| | G-MS- | G-MS- | G-MS- |
|---|---|---|---|
| | lowAw- | lowAw- | lowAw- |
| | 80/ | 80/ | 80/ |
| | 20_#21 | 20_#22 | 20_#23 |
| Mannitol to granular starch dry | 80:20 | 80:20 | 80:20 |
| weight ratio | | | |
| Pressure (bar) | 50 | 50 | 50 |
| Nozzle (SPRAYING SYSTEM | 1 nozzle | 1 nozzle | 1 nozzle |
| type SK) | 52-27 | 52-27 | 52-27 |
| Mannitol solution solid | 40 | 40 | 40 |
| content(%) | | | |
| Mannitol solution flowrate (kg/h) | 240 | 240 | 240 |
| “extra white” corn starch | 28 | 28 | 28 |
| flowrate (kg/h) | | | |
| Upstream air temperature (°C) | 108 | 103 | 127 |
| (drying of step (a)) | | | |
| Temperature of the static fluid | 100 | 100 | 100 |
| bed (°C) (drying of step (b)) | | | |
| Air Water content for static fluid | 0.6 | 0.6 | 0.6 |
| bed and vibrated fluid bed (g/kg | | | |
| of dry air) (drying and cooling of | | | |
| steps (b) and (c)) | | | |
| Temperature of the vibrated | 75 | 75 | 75 |
| fluid bed hot air (°C) | | | |
| (drying of step (b)) | | | |
| Temperature of the vibrated | 20 | 20 | 20 |
| fluid bed cold air (°C) (cooling of | | | |
| step (c)) | | | |
| Outlet air temperature (°C) | 70 | 63 | 82 |

### 2.2. Granules' Characterization

The granules thus obtained were characterized according to the methods described in section 1.2.

They were also examined by scanning electron microscopy (Quanta 200F, FEI)

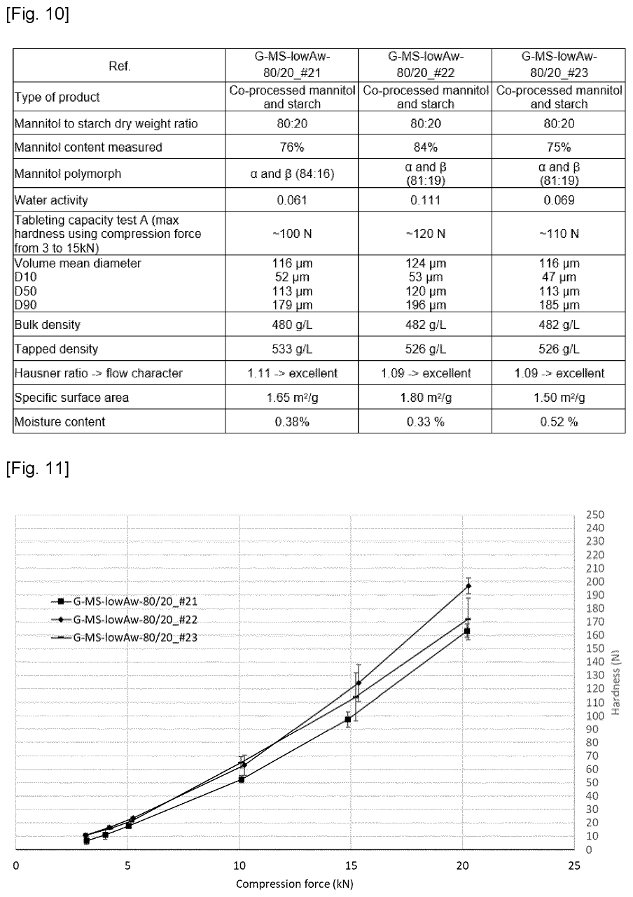

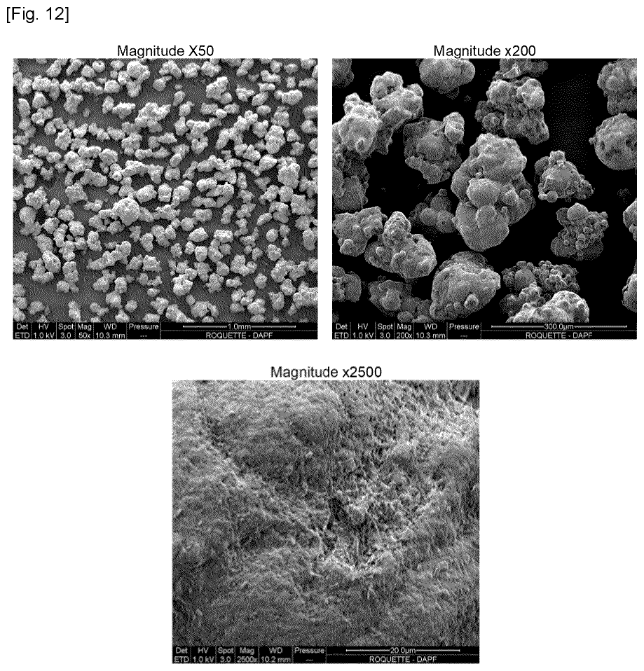

Results are presented in FIGS. 10, 11 and 12.

These results confirm the good tableting capacity of the granules according to the disclosure, as well as the robustness of the process used.

The granules according to the disclosure have excellent flowability (FIG. 10).

Moreover, the taste and organoleptic properties of the tablets obtained were evaluated at chewing. The panel found that the overall taste and feel were good. The tablets were slightly sweet, with no unpleasant aftertaste, and no sandy or chalky mouthfeel.

For further characterization and evaluation, pilot batch G-MS-lowAw-80/20_#22 was selected. Indeed, this batch had the highest Aw, meaning that if the inventors could show that it was good for formulating probiotics, it could be expected that the other batches would be at least as effective, if not more effective.

### 2.3. Stability of Formulations Comprising the Granules and a Probiotic

Formulation with probiotics (powdery compositions and tablets) were prepared as described in section 1.3, except that the tablets were packaged in aluminum blisters. For making the tablets, compression forces were selected in order to match the hardness obtained previously with the prototype G-MS-lowAw-80/20_#1 using 15 kN and 20 kN compression forces. Such compression forces were of 12.5 kN and 16.5 kN respectively.

Friability of the tablets thus prepared was measured as follow: sample of whole tablets corresponding minimally to 6.5 g was used for the evaluation. The tablets were carefully dedusted over a 1 mm sieve prior to evaluation before weighing the tablet samples and placing them in the drum of the friability tester (PTF 20E, Pharma Test Apparatebau AG). The drum was rotated 100 times and tablets were removed after. Remove loose dust from the tablets as before and weigh the tablets. The percentage loss of mass as a function of the initial mass was determined.

Stability was assayed as in section 1.3.

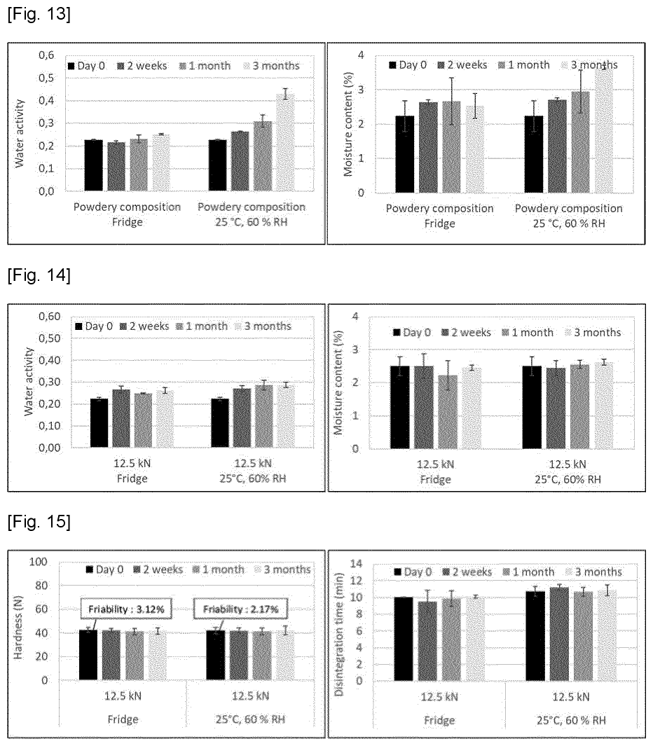

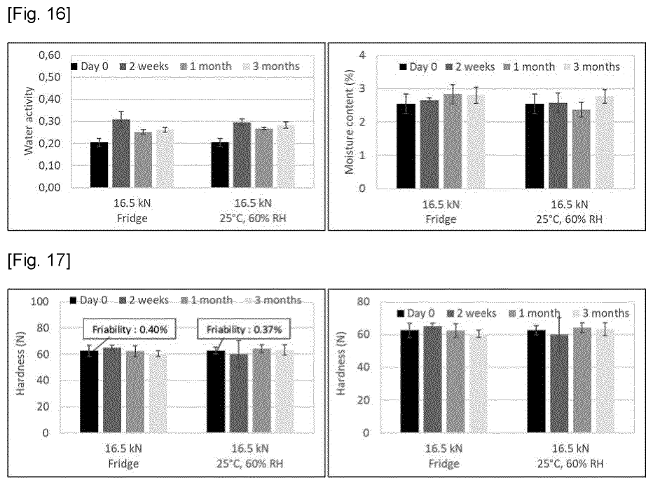

Results are presented in FIGS. 13 to 17.

Given that the initial starting Aw and moisture content of pilot batch G-MS-lowAw-80/20_#22 was 0.111 and 0.33% respectively, the results obtained for the resultant formulations showed significant Aw and moisture contribution from Lb. rhamnosus. This further emphasizes the need for an excipient with low Aw and moisture content in order to maintain an overall acceptable Aw and moisture content of the final formulation.

For powdery compositions, a marginal increase in water activity and moisture was observed. For the fridge samples, water activity was maintained throughout the 3-month storage. Room temperature samples showed a water activity increase of 0.2.

The results obtained for the tablets were notably better. Both water activity and moisture were maintained throughout the 3-month period, for both storage conditions. Moreover, increase in hardness and disintegration time was minimal for both storage conditions and compression forces, which shows the good stability of the tablets obtained from the granules according to the disclosure.

Also, it can be seen that compression force significantly impacts hardness and friability: the lower the compression force, the lower the hardness and the higher the friability.

2.4. Cell Viability in Formulations with Probiotics

Cell viability was assayed in the formulations, in order to evaluate whether the granules according to the disclosure were suitable for formulating probiotics, and more generally moisture-sensitive ingredients. Cell viability was assayed as described in section 1.4.

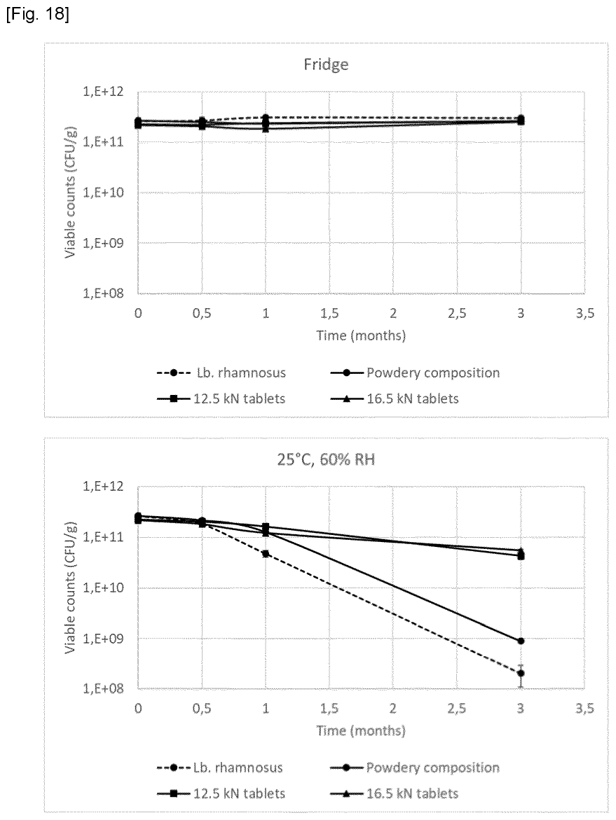

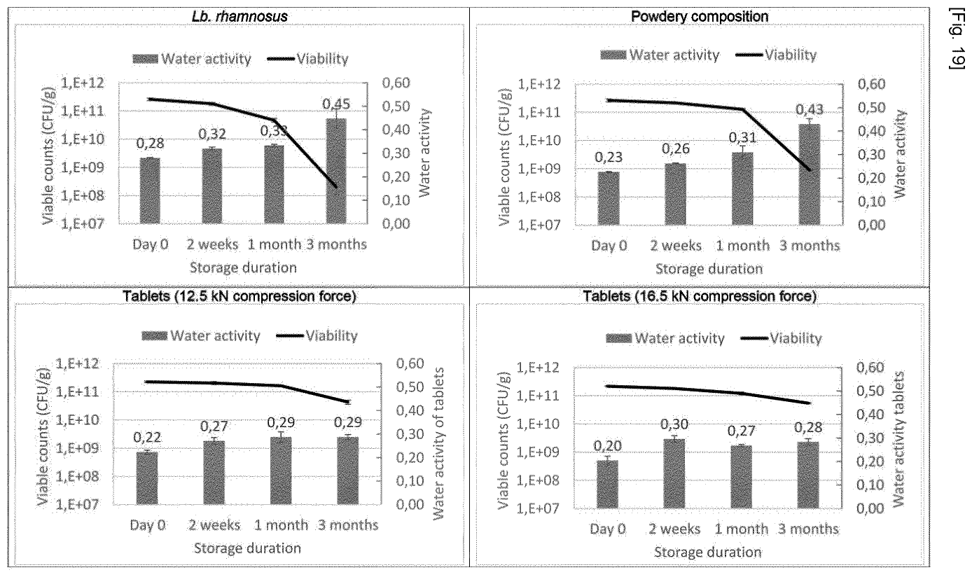

Results are presented FIGS. 18 and 19. On FIG. 19, the inventors plotted the cell viability together with the water activity, for the storage condition 25 °C/60% RH.

FIG. 18 shows that refrigeration was able to maintain the viability of Lb. rhamnosus in all formulations. Unlike standard excipients, this showed that the granules according to the disclosure do not destabilize the probiotics. At room temperature, the granules according to the disclosure even have a protective effect: the decrease in cell viability is less marked in the formulations comprising the granules according to the disclosure and Lb. rhamnosus, as compared to Lb. rhamnosus alone. Cell viability barely decreased when it was formulated into tablets.

When looking at FIG. 19, it can be seen that the cell viability of Lb. rhamnosus alone dramatically decreased, when the water activity increases. By contrast, when combined with the granules according to the disclosure, the cell viability of Lb. rhamnosus is improved. This further highlights the ability of the granules according to the disclosure to limit the increase of free water into probiotics formulas, thus protecting the probiotic from moisture.

3. Comparison with Granules of Starch and Mannitol with Higher Aw (not According to the Disclosure), and with Blend of Mannitol and Starch (not According to the Disclosure).

In order to confirm the importance of having mannitol and granular starch present in same granules (i.e., by being co-processed) and the importance of having a low water activity, the inventors compared granules according to the disclosure with the following comparative samples:
a mixture of directly compressible mannitol (PEARLITOL® 200SD) and of granular starch (maize starch) having a mannitol to granular starch dry weight ratio of 80:20, a Aw of 0.276 and a moisture content of 1.81% (herein after referred to as “blend of mannitol and granular starch having low Aw”); said Aw and moisture content being determined according to the methods given in section 1.3.
a co-processed mannitol and starch according to U.S. Pat. No. 9,839,610, having a mannitol to granular starch dry weight ratio of 80:20, a Aw of 0.545 and a moisture content of 2.32% (PEARLITOL® Flash, herein after referred to as “granules of mannitol and granular starch having high Aw”); said Aw and moisture content being determined according to the methods given in section 1.3.

The comparative blend of mannitol and granular starch having low Aw was obtained by using a grade of granular starch having particularly low moisture content, in order to obtain a blend having the lowest possible moisture and Aw.

### 3.1. Stability of Formulations Comprising the Granules or Blend and a Probiotic

Tablets with probiotics were prepared as described in section 2.3. For making the tablets, compression forces were selected in order to match the thickness obtained with the sampled according to the disclosure (pilot batch G-MS-lowAw-80/20_#22) using 16.5 kN compression force, which was of 6.9 mm.

Stability was assayed as in section 1.3.

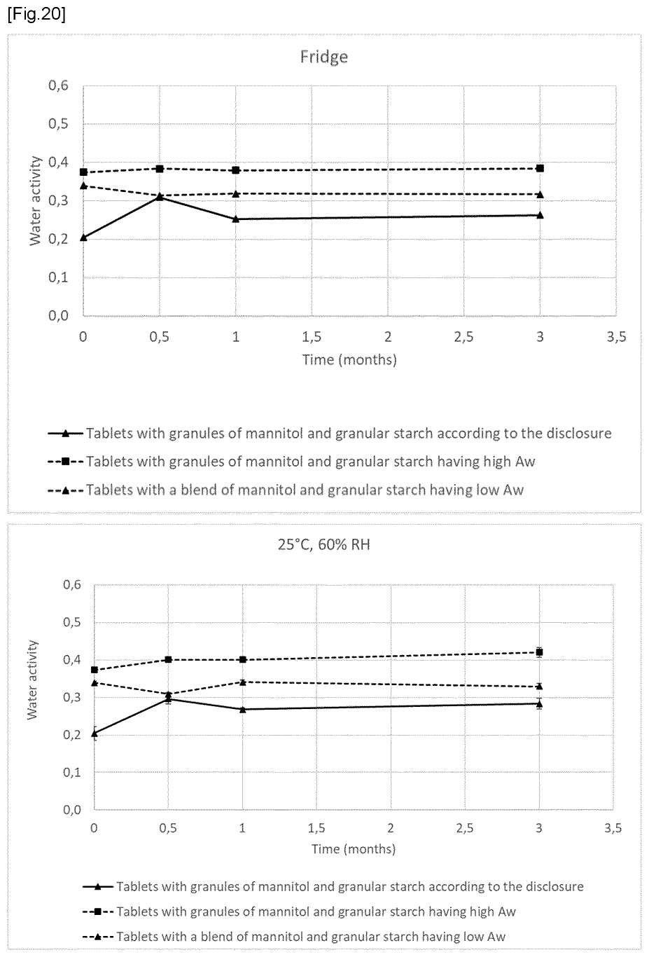

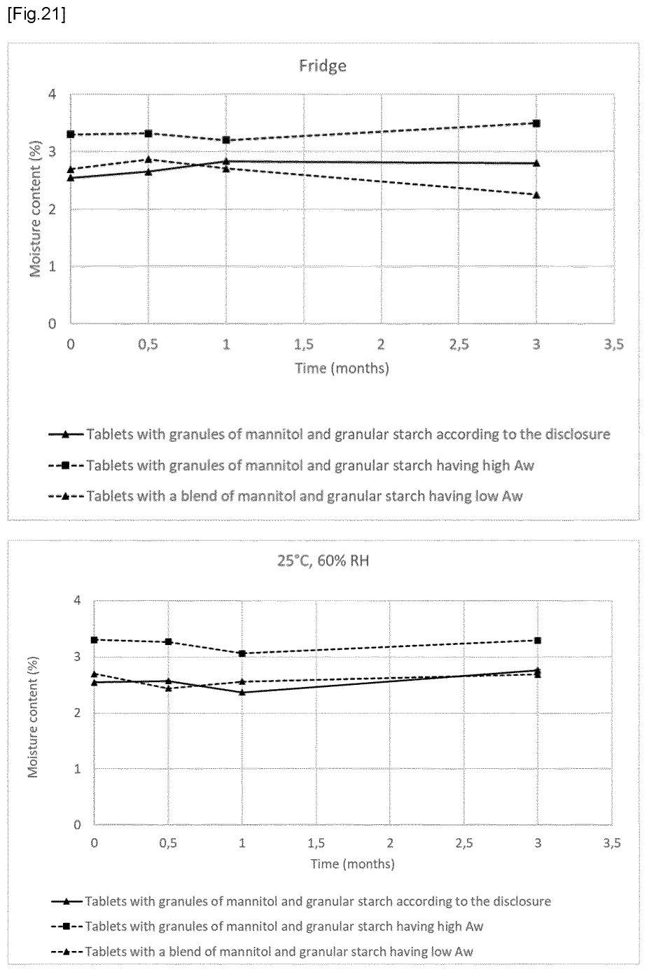

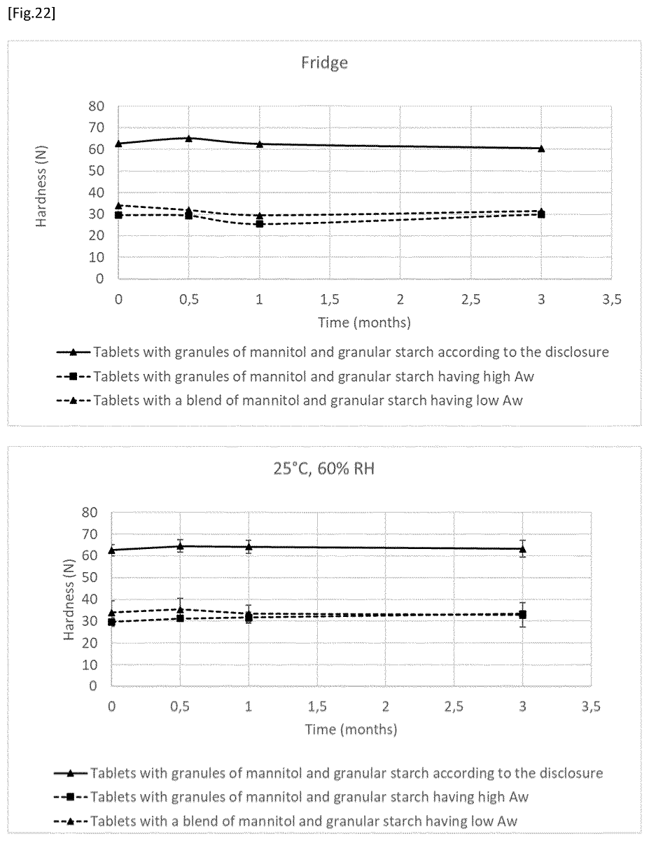

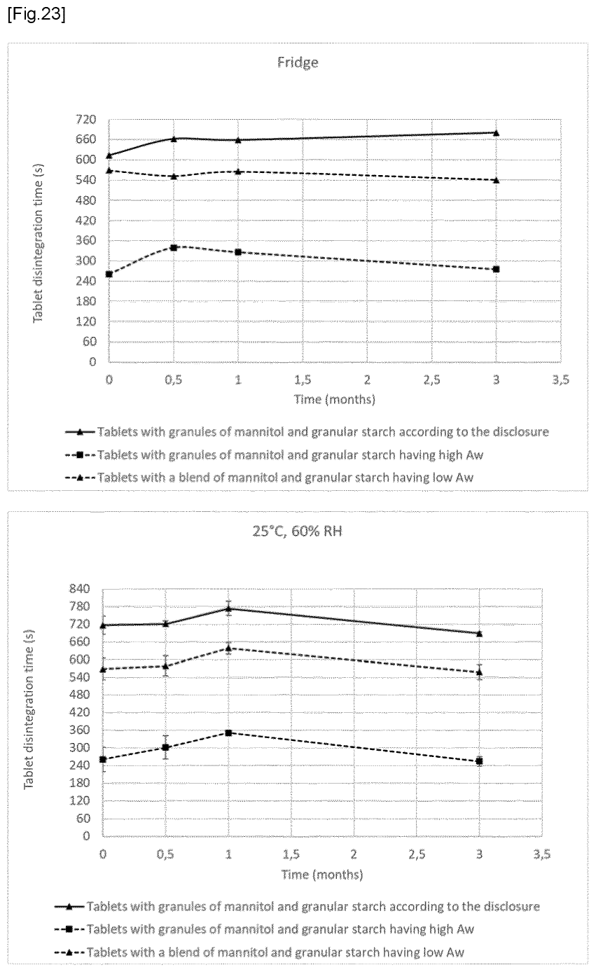

Results are presented in FIGS. 20 to 23.

When compacted to similar thickness, the tablets comprising the granules according to the disclosure were harder, while surprisingly exhibiting the shortest disintegration time.

The hardness and disintegration time of the tablets in both conditions (Fridge and 25 °C/60% RH) were steady over the time, which is a sign of physical stability of the tablets for all three formulations.

The lowest water activity was obtained in both conditions when using the granules according to the disclosure. For these tablets, the water activity of the tablets was maintained in the ideal range of 0.2-0.3 during three months.

3.2. Cell Viability in Formulations with Probiotics

Cell viability was assayed as described in section 1.4.

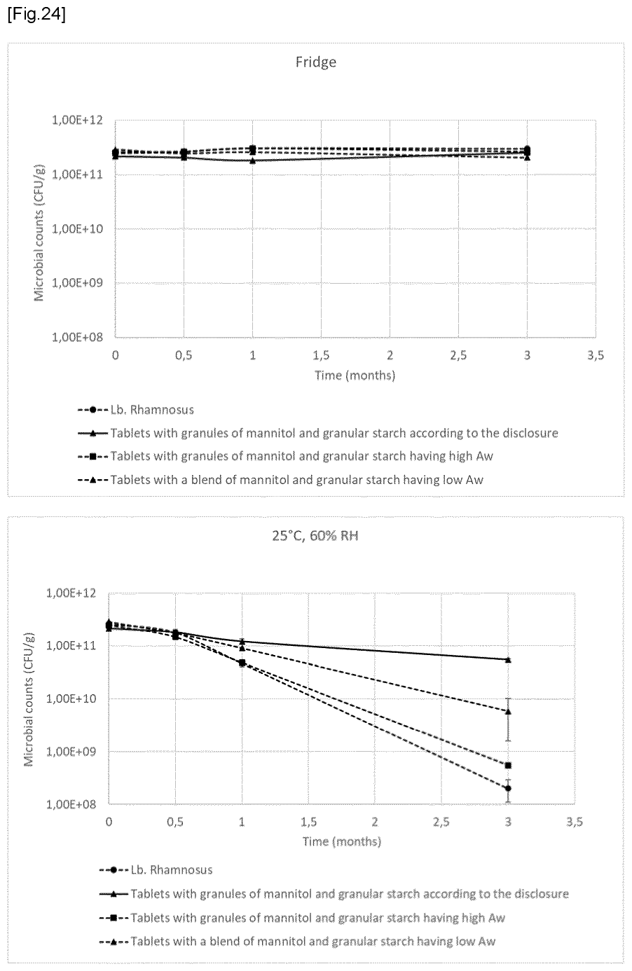

Results are presented FIG. 24.

The granules according to the disclosure showed a remarkable probiotic protective effect. After three months storage at 25 °C/60% RH, the cell viability of the tablets containing the granules according to the disclosure was much higher than the one obtained in the comparative tablets comprising granules having Aw greater than 0.3, or a blend of mannitol and granular starch having low Aw.

These results show that it is essential that the mannitol and granular starch be both present in same granules (i.e., by being co-processed), while exhibiting a low Aw.

1. Granules of microcrystalline mannitol and of granular starch having a water activity equal to or lower than 0.3, said water activity being measured at 25 °C.

2. The granules of claim 1, having water activity equal to or lower than 0.2.

3. The granules of a wherein the dry weight ratio of mannitol to granular starch is equal to or higher than 50:50.

4. The granules of claim 1, having a moisture content equal to or lower than 5.0% by weight.

5. The granules of claim 1, having a bulk density equal to or higher than 350 g/L.

6. The granules of claim 1, having a tapped density equal to or higher than 400 g/L.

7. The granules of claim 1, having a specific surface area equal to or higher than 0.30 m²/g.

8. The granules of claim 1, wherein the mannitol comprises α- and β-polymorphs.

9. The granules of claim 1, having a volume mean diameter equal to or higher than 90 μm, and equal to or lower than 400 μm.

10. A process for making granules according to claim 1, comprising a step (a) of agglomerating microcrystalline mannitol and granular starch by spraying and drying solubilized mannitol onto granular starch, a step (b) of drying the granules obtained in step (a), a step (c) of cooling the granules obtained in step (b), wherein steps (b) and (c) are performed using respectively a drying and a cooling air, said drying and cooling air both having a water content equal to or lower than 3.0 g per kg of dry air.

11. A product comprising the granules according to claim 1, and another ingredient.

12. The product of claim 11, wherein said other ingredient is a moisture-sensitive active ingredient.

13. The product of claim 11, wherein said other ingredient is a probiotic.

14. The product of claim 11, wherein said product is a powdery composition, a tablet or a chewable tablet.

15. A use of the granules of claim 1, for formulating or stabilizing a moisture-sensitive active ingredient.
